(19) United States
(12) Patent Application Publication
(10) Pub. No.: US 2022/0276545 A1
(43) Pub. Date: Sep. 1, 2022

(54) METHOD AND SYSTEM FOR EFFICIENTLY DISINFECTING N95 MASK(S) WITH UPCONVERTING NANOPARTICLES, AND DISINFECTED MASK(S)

(71) Applicant: THE TRUSTEES OF COLUMBIA UNIVERSITY IN THE CITY OF NEW YORK, New York, NY (US)

(72) Inventors: PETER JAMES SCHUCK, New York, NY (US); EMMA ZEYAN XU, New York, NY (US); CHANGHWAN LEE, New York, NY (US)

(21) Appl. No.: 17/682,053

(22) Filed: Feb. 28, 2022

Related U.S. Application Data
(60) Provisional application No. 63/154,358, filed on Feb. 26, 2021.

(51) Int. Cl. G02F 1/35; G02B 5/20; G02F 1/355; A61L 2/08; A61L 2/10; C01F 17/36

(52) U.S. Cl. CPC G02F 1/353; G02B 5/208; G02F 1/3551; A61L 2/085; A61L 2/10; C01F 17/36; G02F 2202/36; G02F 2203/11; A61L 2202/26

(57) ABSTRACT

Exemplary wearable device(s) (e.g., mask(s)) according to exemplary embodiments of the present disclosure can be provided. For example, the wearable device(s) can comprise an inner layer, an outer layer and a filter layer, and The inner layer, the outer layer and/or the filter layer can include one or more upconverting nanoparticles. Further, exemplary method(s) according to the exemplary embodiments of the present disclosure can be provided for disinfecting a wearable device. The exemplary method(s) can comprise, e.g., providing the wearable device comprising an inner layer, an outer layer and a filter layer, in which the inner layer, the outer layer and/or the filter layer can include one or more upconverting nanoparticles. In the exemplary method, it is possible to apply infrared radiation (IR) to the upconverting nanoparticle(s) to generate ultraviolet (UV) radiation so as to disinfect the wearable device.

### CROSS REFERENCE TO RELATED APPLICATION(S)

This application relates to and claims priority from U.S. Provisional Patent Application Ser. No. 63/154,358, filed Feb. 26, 2021, the disclosure of which is incorporated herein by reference in its entirety.

### FIELD OF THE DISCLOSURE

The present disclosure is directed to methods and systems for efficiently disinfecting mask(s) (e.g., N95 masks) with upconverting nanoparticle(s), as well as to mask(s) (e.g., N95 masks) which have been disinfected using upconverting particle(s).

### BACKGROUND INFORMATION

Personal Protective Equipment (PPE) - such as face masks, gowns and gloves - can effectively protect medical staff when they are in contact with patients. To avoid cross contamination, most PPE are discarded after only a one-time use. However, this high burn rate proved to be unsustainable during a global pandemic. During Covid-19, when a large number of patients arrived at the hospital at once, there was simply not enough supply to ensure safe and sterile medical practice. [See, e.g., Ref. 1] PPE shortages force doctors and nurses to attend to patients while insufficiently protected, thus making them the victims of the disease unnecessarily. Since the medical professionals were not getting the sufficient PPE, it was even more difficult for the rest of the population to acquire masks. They suffered from a higher rate of viral transmission than they would have should they have the proper PPE. [See, e.g., Ref. 2]. This, in turn, resulted in more patients at the hospital, and thus created a disastrous and endless cycle of more people getting sick, and fewer available and healthy caretakers. In addition to the paramount importance in health, a large quantity of waste created by PPE is also unsustainable for the environment. There is a need for a solution that can, e.g., ensure the well-being of the population, as well as minimizing the waste.

As manufacturers ramp up their efforts to produce more PPE with higher throughput, scientists are also exploring ways to extend their usage time, while ensuring that they are still sterile. In August of 2020, The Center for Disease Control and Prevention (CDC) endorsed three types of most promising PPE disinfection methods: ultraviolet germicidal irradiation (“UVGI”), vaporous hydrogen peroxide, and moist heat. [See, e.g., Ref 3]

For example, to disinfect masks with vaporous hydrogen peroxide, the masks are first placed within individual sterilization pouches, and then they are placed inside a commercial H₂O₂ vaporization chamber, where 59% H₂O₂ liquid is vaporized. A standard cycle lasts 28 minutes and consists of three stages: conditioning, decontamination, and aeration. [See, e.g., Ref. 4]. While this method has been widely established in hospitals for sterilization, in the particular case of N95 masks, a study has found that depending on the model of the mask, the structural integrity declines after 1-4 cycles, thus making the masks less effective for the user. [See, e.g., Ref. 5].

Another disinfection method has been described in which the masks are exposed to dry heat for an extended period of time. Using such method, masks can be hung inside an incubator and are exposed to 70 degrees Celsius heat. Although this is probably the cheapest and the safest option for the operation personnel, study shows that it takes 60 minutes to reduce the virus amount by log-3 (1000 fold). [See, e.g., Ref 6]. This is a much longer time compared to the other two methods described herein. Additional time is also needed for the masks to cool down before re-usage. A more recent study has shown that the consistency of viral inactivation is questionable, [see, e.g., Ref. 7] hence the efficacy of the dry heat method has to be reevaluated.

UVGI disinfection may require a source of UV light. It is believed that there are two types of UV light that are germicidal, categorized by their range of wavelength: UVB (280-315 nm), and UVC (100-280 nm). [See, e.g., Ref 8]. The disinfection process usually involves a chamber, with a mercury lamp mounted inside to illuminate UV light. The standard lamp produces photons at 254 nm. This is within the UVC range, and can effectively break off the bonds of DNA and RNA inside a virus cell, so they cannot reproduce. [See, e.g., Ref 9]. One drawback of this method, can be that UV is also capable of deteriorating the material that mainly consists of a N95 mask, polypropylene (PP), thus limiting the number of reuses before the mask loses its effectiveness. Due to the intrinsic “shadowing” effect built in the multi-layer mask structure, relatively high intensity of illumination is needed for the photons to fully penetrate the mask layers, and successfully disinfect the virus at all parts of the mask. one study shows that under a 254 nm mercury lamp, some masks degrade significantly when exposed to dosages more than 200 J/cm², thus limiting the number of times they can be recycled. [See, e.g., Ref. 10].

Accordingly, there may be a need to address and/or at least partially overcome at least some of the prior deficiencies described herein.

### SUMMARY OF EXEMPLARY EMBODIMENTS

Such issues and/or deficiencies can at least be partially addressed and/or overcome using, exemplary methods and system, which are described herein for mask manufacture and disinfection. For example, by integrating upconverting nanoparticles (“UNCPs”) into masks, it is possible to disinfect the masks by exposing them to near-infrared (NIR) light.

To that end, exemplary wearable device(s) (e.g., mask(s)) according to exemplary embodiments of the present disclosure can be provided. For example, the wearable device(s) can comprise an inner layer, an outer layer and a filter layer, and The inner layer, the outer layer and/or the filter layer can include one or more upconverting nanoparticles. Further, exemplary method(s) according to the exemplary embodiments of the present disclosure can be provided for disinfecting a wearable device.

In an exemplary embodiments of the present enclosure, the inner layer, the outer layer and/or the filter layer can further include an ultra-violet (UV)-absorbing lanthanide complex material. The UV-absorbing lanthanide complex material can be configured to absorb UV radiation and/or down-convert the UV radiation back to infrared (IR) light. According to further exemplary embodiments, the upconverting nanoparticle(s) can include a NaYF4 hexagonal host lattice and/or can be doped with Lanthanide ions. The Lanthanide ions can include a sensitizer to absorb near infrared photons and an emitter to send out photons at higher energies. The sensitizer can be Yb3+, and the emitter can include Er3+ or Tm3+.

According to additional exemplary embodiments of the present disclosure, the inner layer and/or the outer layer can include non-woven polypropylene. Further, e.g., the filter layer can be or include a meltblown filtration layer. The upconverting nanoparticle(s) can be provided in the filter layer. The UV-absorbing lanthanide complex material can be provided in the filter layer.

In still another exemplary embodiment of the present disclosure, the exemplary method(s) can comprise, e.g., providing the wearable device comprising an inner layer, an outer layer and a filter layer, in which the inner layer, the outer layer and/or the filter layer can include one or more upconverting nanoparticles. In the exemplary method, it is possible to apply infrared radiation (IR) to the upconverting nanoparticle(s) to generate ultraviolet (UV) radiation so as to disinfect the wearable device.

In various exemplary embodiments of the present disclosure, the IR radiation can include the near-IR radiation. The inner layer, the outer layer and/or the filter layer can further include an ultra-violet (UV)-absorbing lanthanide complex material. The exemplary embodiments of the method can include absorbing the UV radiation by the UV-absorbing lanthanide complex material upon the impact of the UV radiation on the wearable device. In further exemplary embodiments of the present disclosure, it is possible to down-convert the UV radiation by the UV-absorbing lanthanide complex material into a subsequent IR radiation upon the impact of the UV radiation on the wearable device. The UV-absorbing lanthanide complex material is provided in the filter layer. Further, the upconverting nanoparticle(s) can be provided in the filter layer. Additionally, one or more the upconverting nanoparticles can be doped with Lanthanide ions.

Exemplary methods and systems according to various exemplary embodiment of the present disclosure provide efficient disinfection that can lengthen the lifetime of mask usage significantly, as well as operate at wavelengths that are more human friendly.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF DRAWINGS

Further objects, features and advantages of the present disclosure will become apparent from the following detailed description taken in conjunction with the accompanying Figures showing illustrative embodiments of the present disclosure, in which:

FIG. 1A is an illustration of the effect of UV photon breaking down DNA;

FIG. 1B is an illustration of the effect of UV photon breaking down polymer chain;

FIG. 1C is an exemplary application of the conventional UV radiation and Far-UVC radiation on skin layers and eyes, illustrating that Far-UVC does not penetrate through the top layer of the skin and the eyes;

FIG. 1D is an exemplary diagram of an upconverting nanoparticle (“UCNP”) converting near-infrared (“NIR”) radiation or light into UV according to an exemplary embodiment of the present disclosure;

FIG. 2A is a set of illustration of outer layers of an exemplary N95 mask;

FIG. 2B is a diagram showing a comparison of a shadowing effect of infra-red (“IR”) radiation versus ultraviolet (“UV”) light;

FIG. 2C is a diagram of an exemplary interaction between UCNP, the virus, and lanthanide complex on a single sheet of PP fabric, according to an exemplary embodiment of the present disclosure;

FIG. 2D is a diagram of the UCNP and lanthanide complex embedded in an exemplary N95 mask, according to an exemplary embodiment of the present disclosure;

FIG. 3A is a graph of a UCNP emission in the far UVC spectrum, according to an exemplary embodiment of the present disclosure;

FIG. 3B is an exemplary energy diagram of NaYF4 host matrix and Lanthanides that can emit in the far UVC region, according to an exemplary embodiment of the present disclosure;

FIG. 4A is a set pf graphs the illumination time needed per mask, when varying laser power, UCNP mask volume, and quantum yield of UCNPs according to exemplary embodiments of the present disclosure;

FIG. 4B is a graph of the cost of UCNP likely needed per mask at different production scale;

FIG. 4C is a graph of an approximate cost when buying 1-1000 masks; and

FIG. 5 is a flow diagram of a method for disinfecting mask(s) in accordance with the exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended claims.

### DETAILED DESCRIPTION OF EXEMPLARY EMBODIMENTS

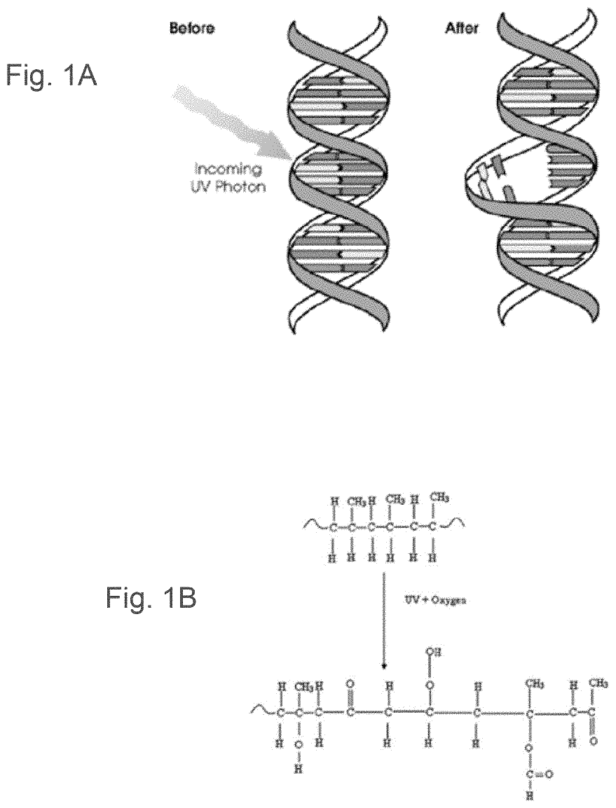

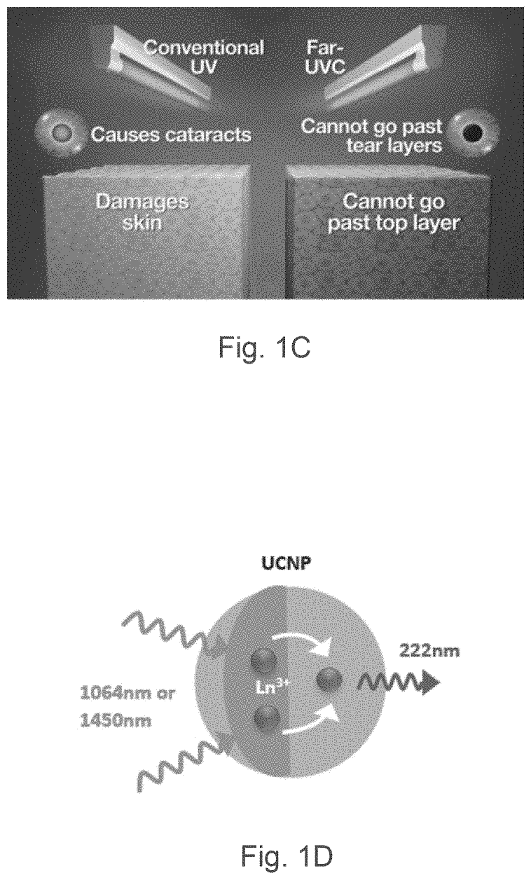

FIG. 1A shows an illustration of the effect of UV photon breaking down DNA, FIG. 1B illustrates the effect of UV photon breaking down polymer chain, and FIG. 1C shows an exemplary application of the conventional UV radiation and Far-UVC radiation on skin layers and eyes. As illustrated in FIG. 1C, conventional UV radiation, when applied to the eyes and skin causes cataracts and damages skin, respectively. In addition, as provided in FIG. 1C, the application of Far UVC radiation on the eyes and skin, does not penetrate tear layers, and cannot proceed past the top layer of the skin, respectively.

To address such issues, as shown in FIG. 1D, it is possible to utilize one or more upconverting nanoparticles (“UCNPs”) to convert NIR radiation or light (e.g., at approximately 1064 or 1450 nm wavelength(s) into UV radiation or light (e.g., at about 222 nm wavelength) according to an exemplary embodiment of the present disclosure.

FIG. 1D illustrates that UCNPs can have a NaYF₄ hexagonal host lattice, which can be doped with Lanthanide ions that may be responsible for photon upconversion. For example, two or more different lanthanide ions can be used, i.e., (i) a sensitizer to absorb the incident NIR photons, the most common one can be Yb³⁺, and (ii) an emitter to send out photons at higher energies, such as Er³⁺ or Tm³⁺. [See, e.g., Ref 11]. Certain common applications for UCNPs can include engineering them for in-vitro bioimaging, since NIR exposure can be much less harmful for cells, [See, e.g., Ref. 12] and has a much higher penetration depth compared to other visible probes. [See, e.g., Ref. 13].

It has been investigated a conversion of the NIR to UV conversion of UCNPs for various applications such as lasing [see, e.g., Ref 14] and solar cells [see, e.g., Ref 15]. One study demonstrates a five-photon upconversion process of UCNPs doped with Thulium (Tm) and Gadolinium (Gd). It was possible to achieve bright emission at 290 nm and 311 nm for Tm and Gd, respectively. [See, e.g., Ref. 14]. These UV wavelengths are within UVB region, and thus, can be germicidal. Similar emissions has also been found with lanthanide atoms doped in different crystal lattice structure such as YF₃ [see, e.g., Refs. 15 and 16] and LiYF4 [see, e.g., Ref 17]. These studies assist with the understanding the physics behind NIR to UV photo upconversion process, and facilitate various directions for enhancing these nanoparticles for application specific designs for the future.

Since UCNPs can, for example, provide an extremely local source of UV emission, according to an exemplary embodiments of the present disclosure, it is possible to embed UCNPs in, e.g., N95 masks and other masks. For example, embedding UCNPs in one or more polymers can be an appropriate process, and has been demonstrated in various studies.

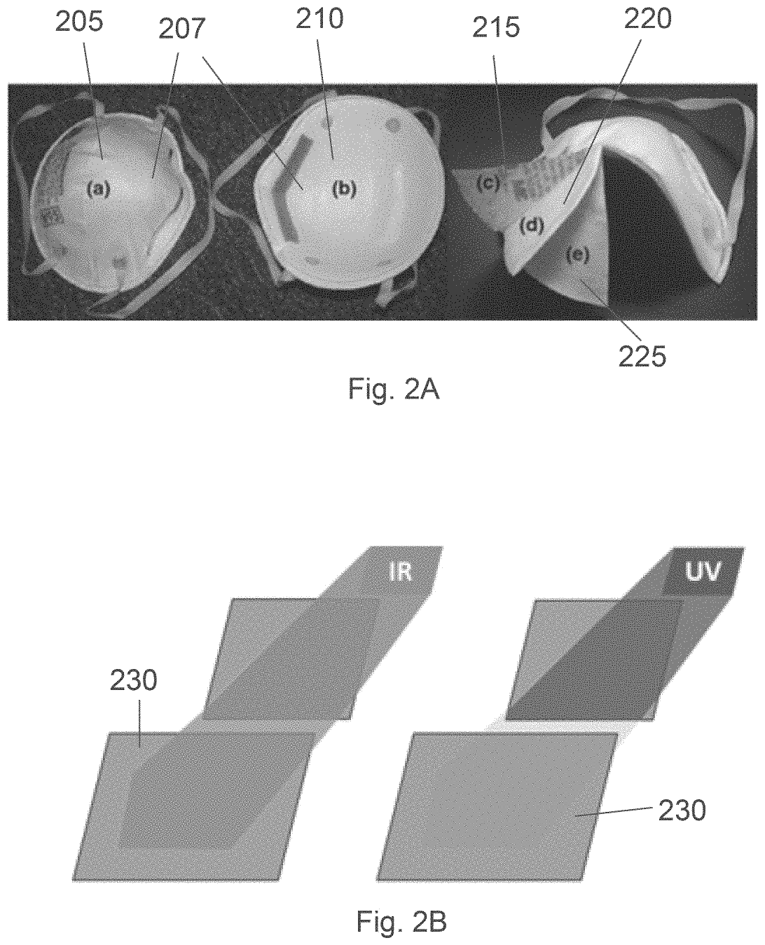

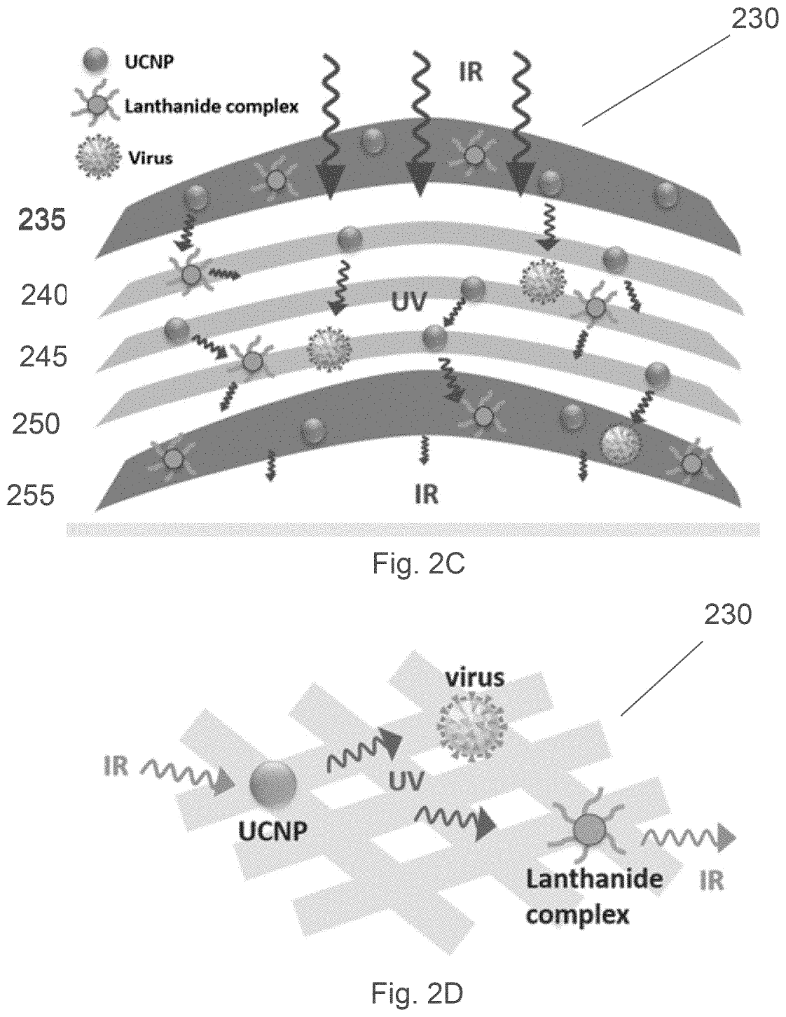

For example, FIG. 2A shows a set of illustrations of the layers of a typical N95 mask. For example, the exemplary mask can include an environment facing (or front) layer, and a metal nose piece 207 which is attached on the top part of the mask, to ensure a good fit. In addition, a layer 210 can be provided that is configured to directly contact the user, which can be or include a soft piece of foam fabric is attached in the nose piece 207 or area to increase comfort. Further, as shown in FIG. 2A, meltblown filtering layers 215, 220, 225 can be provided for the mask.

According to an exemplary embodiment of the present disclosure, it is possible to embed UCNPs in one, some or all of the mask layers to facilitate an appropriate or maximum disinfection coverage. Upon incident of NIR light/radiation and/or beam thereon, the UCNPs can subsequently convert the NIR photons into UV photons, and disinfect the masks internally. This exemplary configuration can facilitate the irradiation of significantly fewer UV photons, compared to the illumination intensities used in the past for disinfecting. One of the reasons why fewer UV photons may be needed can be that the other approaches may require using enough UV power to penetrate to the center of the mask layers, and overcome shadowing effects. To address such possible prior deficiencies, the UCNP disinfection system and method according to the exemplary embodiment of the present disclosure reduces and/or minimizes the impact of UV degradation of the mask, as well as provided for a lower disinfecting illumination power, among other benefits.

For example, according to an exemplary embodiment of the present disclosure, an illumination chamber that emits NIR light can be provided, instead of UV, which can have various benefits. There can be less degradation on the chamber, hence it can facilitate a cheaper material and process for the chamber manufacturing. NIR can also be much safer for a human operation in close proximity. This exemplary disinfection method according to the exemplary embodiment of the present disclosure can be utilized for an active disinfection. That is, the exemplary disinfection process can occur while the mask is still worn on the face of the patient or doctor. This can be effectuated because of, for example, the low energy of NIR photons and the weak penetration ability of far UVC photons, both of which are of little to no harm to the human body.

There may be some issues associated with the UVGI disinfection. For example, the UV generated by UCNPs may deteriorate the PP fibers of the mask. To address this problem, according to an exemplary embodiment of the present disclosure, it is possible to add a UV-absorbing lanthanide complex material to be embedded with the UCNP together within the mask. The lanthanide complex can absorb UV radiation/light, and down-convert it back to IR radiation. [See, e.g., Ref. 18]. FIG. 2B illustrates a diagram of an exemplary mechanism effectuated on a single sheet of the mask fiber. For example, as shown in FIG. 2B, when IR radiation/light impacts the mask 230, the UCNP can convert it into UV germicidal photons to, for example, kill the virus. The leftover UV photons can encounter a nearby lanthanide complexes particle, and can be subsequently converted back into IR photons before exiting the mask 230.

FIG. 2C shows a diagram of an exemplary implementation of the mask according to an example embodiment of the present disclosure. As shown in FIG. 2B, e.g., grey layers 235, 255 can be or include the non-woven polypropylene on the outer (environment facing) and inner (user facing) sides of the mask, the green layers 240, 245, 250 can be the meltblown filtration layers, which can be thin and efficient in filtering out micron-size virus particles. FIG. 2D illustrates a diagram of the UCNP and lanthanide complex embedded in an exemplary mask 230 (e.g., N95 mask), according to an exemplary embodiment of the present disclosure As shown in FIGS. 2C and 2D, UCNPs and lanthanide complex particles can be embedded anywhere and everywhere within the mask. UV light may only exist within the mask for disinfection, the incoming and the outgoing photons may only be in the IR range and spectrum. Embedding UCNPs into polymers has previously been demonstrated in various systems [see, e.g., Refs. 19, 20, 21, and 22], thus integrating UCNP into PP fibers is reasonably easy to achieve. For example, the meltblown filter layers 240, 245, 250 can be manufactured at a temperature between 215-340 Celsius, depending on the material. [See, e.g., Ref 23]. This temperature range likely may not pose any threat to physical or chemical alternations of UCNPs, thus UCNPs can be safely integrated into the melt-blown process for the manufacturing of the filter layers.

While UCNPs can be securely locked in the PP fibers during the mask manufacturing process, the biosafety of UCNPs in the unlikely case of their accidental inhalations can also be considered. It is believed that there is currently no literature on the inhalation of UCNPs. However, as UCNPs have applications in biological studies—including bioimaging and drug delivery—researchers have investigated their toxicity via in-vivo studies of mice. In general, depending on the size and surface chemistry, most UCNPs find their way out of the mice through feces in a matter of days. The level of toxicity is also dosage dependent. [See, e.g., Ref. 24]. However, given the fact that UCNP can be locked safely inside the PP fibers, their inhalation would be unlikely, it can be preliminarily concluded that UCNPs embedded in masks can be safe for users.

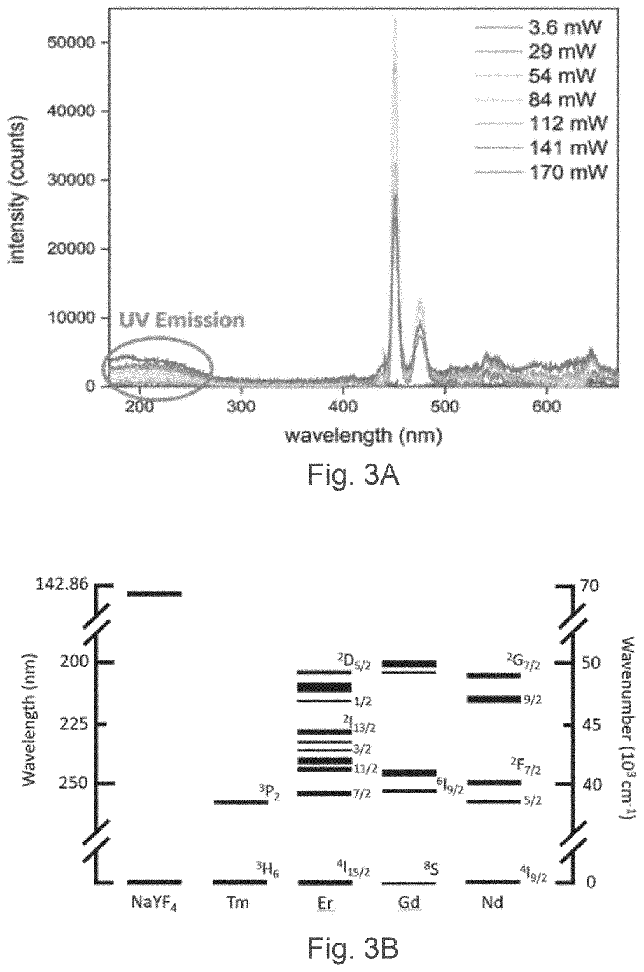

There are studies on the necessary UVGI dosage for mask disinfection. Although the dosage depends on the specific virus, and there has not yet been a study for SARS-CoV-2, studies on similar viruses can be used as references. As a 3-log reduction in virus can be considered significant post treatment, one study showed that a dosage of 1 J/cm2 of 254 nm UVC light was needed to disinfect masks contaminated with H1N1 influenza. [See, e.g., Ref 25]. Importantly, however, by shifting to shorter wavelength far UV-C excitation using 222 nm photons, a study indicates that the appropriate dosage can be, e.g., around 1.5 mJ/cm² to achieve a 3-log reduction. [See, e.g., Ref 26]. In a previous study, avalanching nanoparticles (“ANPs”) that exhibit extremely nonlinear responses have been discussed. [See, e.g., Ref. 27]. These ANPs (or UCNPs) have another important property as shown in the photoluminescence spectrum of the exemplary graph in FIG. 3A, which emit in a broadband UVC region, which includes far-UVC. Thus, it is possible to utilize the far-UVC dosage for the subsequent calculations/determinations.

For example, according to an exemplary embodiment of the present disclosure, to calculate or otherwise determine the quantity of nanoparticles needed to achieve such a dosage and mask design, it is possible to first determine the energy of one 222 nm photon to be 8.95E-19 J. At a dosage of about 1.5 mJ/cm², the corresponding number of far UVC photons is 1.68E+07. To calculate/determine how many NIR photons are needed to produce the number of UV photons needed, it is possible to consider the quantum yield (QY) of such conversion, that is, how efficiently can an exemplary system convert the incident light into outgoing light. Although currently there is no study on the QY of UCNP for the IR to far UVC conversion, related literature can be informative. A recent study demonstrated a core-shell-shell structure, with Yb³⁺ ions in the core, Er³⁺ ions in the inner shell, and an additional inert shell on the outside to encapsulate and prevent surface energy losses. QY was achieved as high as 15% in the visible light spectrum. [See, e.g., Ref. 28]. Thus, it is possible that with an exemplary nanoparticle engineering, the QY of IR to far UVC conversion can be within 1-10%.

UCNPs' ability to convert light can largely depend on an exemplary absorption cross section area thereof. This exemplary area can be determined by, e.g., the absorption cross section area of the Yb³⁺ ion, multiplying by the number of such ions in a UCNP. an exemplary study shows that the absorption cross section of Yb³⁺ ions at 980 nm is 1E-20 cm². [see, e.g., Ref. 29]. Following the UV emissions of UCNP paper [see, e.g., Ref. 14], where the diameter of the nanoparticles is 30 nm, it is possible to calculate that there are approximately 106 Yb³⁺ ions per UCNP. Thus, the absorption cross section per UCNP can be, e.g., 1E-18 cm².

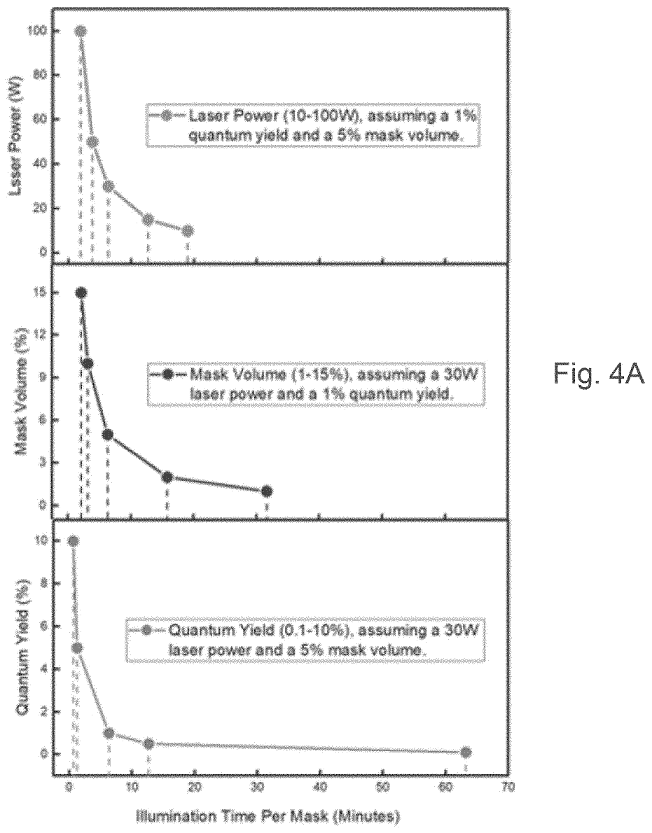

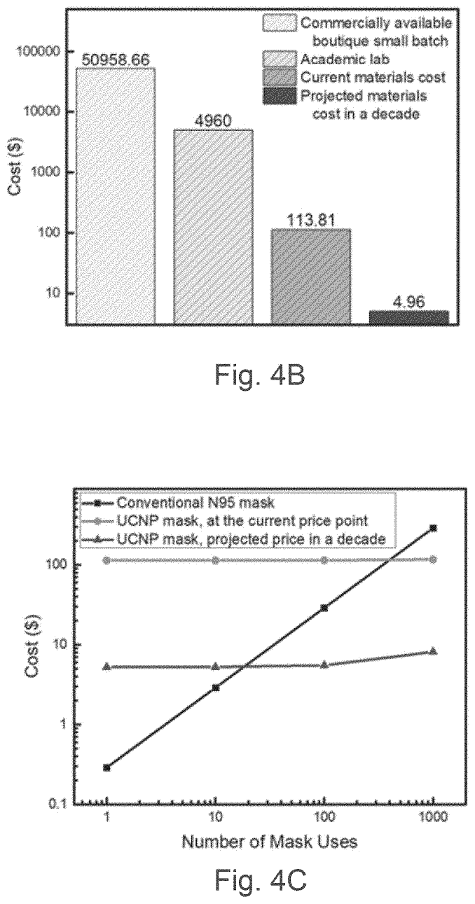

Then, it is possible to calculate or otherwise determined the illumination time needed to disinfect one N95 mask. For example, it is possible to utilize 100 cm² as the surface area of the mask and/or identify, e.g., 3 main variables: (i) the incident laser power (approx. 10-100 W), (ii) the percentage of the mask volume that comprises of UCNPs (e.g., 1-15%), and (iii) quantum yield of the UCNPs (approx. 0.1-10%). The top curve in FIG. 4A varies the laser power while assuming a 1% QY and a 5% UCNP content in the mask. The middle curve varies the UCNP mask volume while assuming a 30 W laser power and a 1% QY. The bottom curve varies the QY of UCNPs, while assuming a 30 W laser input and a 5% mask volume.

One exemplary way to further improve the efficiency of disinfection is to provide, e.g., more efficient particles. It is possible to identify energy transfer pathways between the host matrix, the sensitizer and the emitter lanthanide ions. FIG. 3B shows an exemplary energy diagram of NaYF₄ host matrix and Lanthanides that can emit in the far UVC region, according to an exemplary embodiment of the present disclosure. For example, NaYF₄ has a bandgap of about 8.5 eV (e.g., 68.5 103 cm⁻¹). In FIG. 3b, the bandgap of the host matrix NaYF₄, along with the energy levels of lanthanide ions. 50000 cm⁻¹ corresponds to the wavelength of 200 nm. While Yb³⁺ ions may be the designated sensitizer in almost all UCNP systems, the emitter ion can be selected among several candidates, e.g., Tm³⁺, Gd³⁺, Nd³⁺, Er³⁺. They can have energy levels that extends far in the UVC region and compatible with the exemplary design of the exemplary embodiment of the present disclosure.

For example, in other UVGI disinfection studies, the illumination time can be approximately 30 minutes [see, e.g., Ref. 30], in comparison, most of the data points in FIG. 4A indicates shorter disinfection times, and thus making the disinfection method of the present disclosure more efficient. Indeed, FIG. 4A shows a set of graphs of the exemplary illumination time needed per mask, when varying laser power, UCNP mask volume, and the quantum yield of UCNPs.

It can be useful to determine the cost of UCNP for a single mask. For example, if a 5% of the mask is comprised of UCNPs is assumed, it is possible to obtain 1.19E+17 UCNPs per mask. The molecular weight can be extrapolated to be 1E+07 g/mol. [See, e.g., Ref. 31]. Hence the weight of the UCNP is 1.98 grams. FIG. 4b shows a graph of the cost of UCNP likely needed per mask at different production scale from different sources. The 4th data point is the cost projection extrapolated from the cost of QD. If mass produced, the overhead can be minimized, and the materials cost would only need to be considered, which can be only about $0.57 per 10 mg, and about $113.81 for the whole mask. These numbers are reflected in the graph of FIG. 4B, where the cost in the y-axis is drawn in log-scale to best reflect the dramatic disparity between different scales of production.

To understand these numbers in context and provide reasonable rationale for future cost estimation, it is possible to compare them to the price trajectory of quantum dots. In particular, noting how significant increases in application spaces and demand drove a large scale-up in production capabilities, leading to considerable decreases in costs. Similarly to UCNPs, quantum dots are nanoparticles that can also be used as probes for bioimaging and solar energy harnessing. Moreover, revolutionized electronic display technologies have been revolutionized in the past decade. For example, in 2009, the cost was $3000-$10,000 per gram, similar to UCNPs. [See, e.g., Ref 32]. In 2018, e.g., a cost analysis on quantum dots show that the cost has significantly reduced to $11-$59 per gram. [See, e.g., Ref. 33]. In 2020, that number has been further reduced to a mere $8.11 [see, e.g., Ref. 34]—representing a drop in costs by 3 orders of magnitude over one decade. Currently, UCNPs are still produced in relatively small, boutique batches. If they were to follow the same cost reduction trend as quantum dots, the $113.81 material cost could shrink to $0.11 in a decade. This is reflected as the 4^{th} data point shown in FIG. 4B. For example, despite the cost of quantum dots shrinking over the years, however, when purchasing them today from Sigma-Aldrich, it would still likely cost $2900 per gram, highlighting that retail prices at chemical companies are not accurate reflections of the actual cost of the material.

Each of the masks according to the exemplary embodiments of the present disclosure can be reused many more times than the conventional N95 mask. For example. FIG. 4C shows a graph of the cost projection for reusing the UCNP-embedded masks up to 1000 times vs. purchasing 1000 disposable masks. In the past, the CDC recommended each mask to be worn no more than 5 times, to ensure that the mask remains a good fit for the wearer. [See, e.g., Ref 3]. The relevant study shows that when worn more than 5 times, the straps and the nose pieces will no longer hold the mask in place. [See, e.g., Ref. 35]. Thus, for these calculations, it is possible to factor in 1 set of new straps and nose piece for every 5 reuses. At current UCNP costs, e.g., when the UCNP masks are reused for 400 times or more, it is less expensive than buying 400 conventional N95 masks. The price difference can be more pronounced with more numbers of reuse. As the UCNP costs fall, the number of reuses required to “break even” relative to purchasing conventional N95 masks also fall proportionally. In particular, following quantum dot cost trends, As illustrated in FIG. 4C, only 20 reuses are needed to make the UCNP-N95 masks more cost-efficient (including the replacement of nose pieces and straps every 5 reuses). Moreover, the projected exemplary cost in a decade can be $5 for ˜200 reuses, which we believe is a reasonable goal for future mask use.

For example, the limiting factors of mask re-usage to be physical damages such as tearing or staining. While another limitation may be NIR photodamage, it is unlikely to play a significant role. According to one study that characterized the optical absorption of PP powder, it is shown that the absorbance of UVC is 10 times that of NIR. [See, e.g., Ref. 36]. UV has been shown as providing its photodegradation effect on PP, which compromises the structural integrity of PP irreversibly. [See, e.g., Ref. 10]. Meanwhile, there is no record of NIR having any significant degradation effects. In addition, an exemplary calculation shows that under the NIR treatment, the temperature of the mask would raise by 5.3° C., this is an insignificant or small increase in temperature, as the melting temperature of PP is recorded at 186.1° C. [See, e.g., Ref. 37]. An exemplary design , in one exemplary embodiment of the present disclosure, the UV emission source can be embedded inside the mask, which can significantly decrease the UV exposure of PP, especially at outer surfaces, since the high doses of UV radiation required to penetrate the material and overcome shadowing effects may no longer be required.

If the UV generated by the UCNPs is found to be the ultimate limiting factor of mask reusability, then molecular UV scavengers, e.g., lanthanide complexes as illustrated in FIGS. 2C and 2D, can be also integrated into the polymer. Lanthanide complexes can operate in the opposite manner as UCNPs. For example, upon incident UV photons, these complexes can down-convert the high energy photons into photons with much lower energy. A study showed that by adding lanthanide complexes into PP, an improvement was observed chemically in photodegradation resistance to UV. Additionally, it was found that the mechanical properties of PP was also fundamentally improved for the better. [See, e.g., Ref. 18].

It is known that UV radiation may be harmful to humans and/or animals. For example, the same way as such radiation destroys viruses, it can attack and/or damage the DNA in human cells and possibly cause irreversible damage. [See, e.g., Ref 38]. During the traditional UVGI process, according to the exemplary embodiment of the present disclosure, the entire disinfection process can occur inside a chamber, with subjects (e.g., humans) being advised to maintain a distance, and refrain from looking at the chamber. This problem may not be relevant with the exemplary embodiments of the present disclosure. In particular, with the irradiation source being the relatively harmless and highly penetrable NIR, the exemplary method according to the exemplary embodiments of the present disclosure would be safe for human operation, and it can provide an active disinfection whereas, e.g., the user can wear the mask while the disinfection takes place.

It should be understood that reusing masks is not only economical, it could also be a relief for the environment, as millions of disposable masks are being dumped into landfill each year during normal times. This problem is particularly aggravated by the Covid-19 pandemic. It was reported that 468.9 tons of medical waste was created every day in China alone. [See, e.g., Ref 39]. Additional study suggested that if the global population used one disposable mask per day until the end of the pandemic, there could be a waste as enormous as 129 billion masks. [See, e.g., Ref 40]. This colossal number could potentially be lowered by multiple orders of magnitude if the masks are being disinfected and reused. The disinfection mechanism with IR light source and UCNP described in this disclosure can be a promising candidate to achieve that.

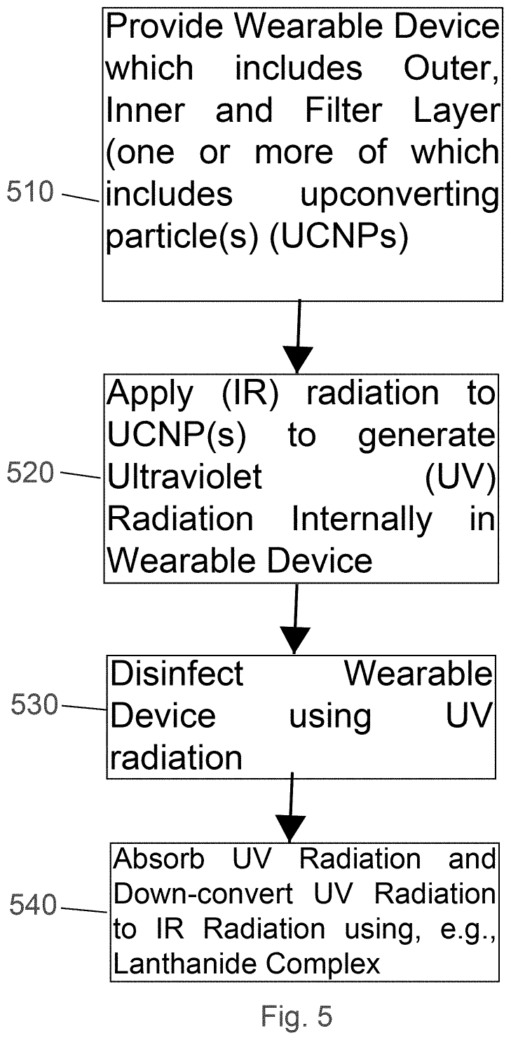

FIG. 5 shows a flow diagram of a method for disinfecting mask(s) in accordance with the exemplary embodiment of the present disclosure. For example, it is possible to provide exemplary method(s) which can disinfect wearable device(s) (e.g., mask(s)) which can include various providing the wearable device(s) in procedure/step 510. In particular, the wearable device comprising an inner layer, an outer layer and a filter layer. The inner layer, the outer layer and/or the filter layer can include one or more upconverting nanoparticles (UCNPs). In procedure/step 520, the IR radiation can be applied to the UCNP(s) to generate UV Radiation internally in the wearable device. Then, the wearable device(s) is thus disinfected using the generated UV radiation in procedure/step 530. Further, in procedure/step 540 the UV radiation is absorbed and/or down-converted to IR radiation using, e.g., lanthanide complex material.

To summarize, exemplary methods to disinfect and reuse N95 masks with UVGI by integrating UCNPs into the masks as a medium, according to the exemplary embodiments of the present disclosure are described herein. For example, the degradation of mask materials during disinfection can be significantly reduced, thus facilitating for more reuse cycles. These exemplary method and system according to the exemplary embodiments of the present disclosure can also be safe for humans in proximity; thus, their exemplary applications is extended and applicable beyond mask disinfections, and be adapted into a variety of environments. In addition to masks, the same or similar principles can also be applied to medical gowns. Moving beyond the medical world, this disinfection framework can be applied to commercial apparel such as certain clothing and accessories as well. From a sustainability point of view, reusing PPE could lead to dramatic decrease in excess waste, thus putting less strain on the environment.

The foregoing merely illustrates the principles of the disclosure. Various modifications and alterations to the described embodiments will be apparent to those skilled in the art in view of the teachings herein. Ilt will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and paragraphs thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
1. Ranney, M. L., V. Griffeth, and A. K. Jha, Critical Supply Shortages—The Need for Ventilators and Personal Protective Equipment during the Covid-19 Pandemic. New England Journal of Medicine, 2020. 382(18): p. e41.
2. Howard, J., et al., An evidence review of face masks against COVID-19. Proceedings of the National Academy of Sciences, 2021. 118(4): p. e2014564118.
3. Implementing Filtering Facepiece Respirator (FFR) Reuse, Including Reuse after Decontamination, When There Are Known Shortages of N95 Respirators. Centers for Disease Control and Prevention, 2020.
4. Ludwig-Begall, L. F., et al., The use of germicidal ultraviolet light, vaporized hydrogen peroxide and dry heat to decontaminate face masks and filtering respirators contaminated with a SARS-CoV-2 surrogate virus. Journal of Hospital Infection, 2020.
5. Lieu, A., et al., Impact of extended use and decontamination with vaporized hydrogen peroxide on N95 respirator fit. American Journal of Infection Control, 2020.
6. Pascoe, M. J., et al., Dry heat and microwave-generated steam protocols for the rapid decontamination of respiratory personal protective equipment in response to COVID-19-related shortages. Journal of Hospital Infection, 2020. 106(1): p. 10-19.
7. Perkins, D. J., et al., COVID-19 global pandemic planning: Dry heat incubation and ambient temperature fail to consistently inactivate SARS-CoV-2 on N95 respirators. Experimental Biology and Medicine, 2020: p. 1535370220977819.
8. Kowalski, W., Ultraviolet Germicidal Irradiation Handbook: UVGI for Air and Surface Disinfection. 2010: Springer Berlin Heidelberg.
9. Lowe, J. J., Katie D. Paladino, Jerald D. Farke, Kathleen Boulter, Kelly Cawcutt, Mark Emodi, Shawn Gibbs et al., N95 Filtering Facepiece Respirator UVGI Process for Decontamination and Reuse. Nebraska Medicine, 2020.
10. Lindsley, W. G., et al., Effects of Ultraviolet Germicidal Irradiation (UVGI) on N95 Respirator Filtration Performance and Structural Integrity. J Occup Environ Hyg, 2015. 12(8): p. 509-17.
11. Gargas, D. J., et al., Engineering bright sub-10-nm upconverting nanocrystals for single-molecule imaging. Nat Nanotechnol, 2014. 9(4): p. 300-5.
12. Nam, S. H., et al., Long-term real-time tracking of lanthanide ion doped upconverting nanoparticles in living cells. Angew Chem Int Ed Engl, 2011. 50(27): p. 6093-7.
13. Tian, B., et al., Low irradiance multiphoton imaging with alloyed lanthanide nanocrystals. Nat Commun, 2018. 9(1): p. 3082.
14. Chen, X., et al., Confining energy migration in upconversion nanoparticles towards deep ultraviolet lasing. Nat Commun, 2016. 7: p. 10304.
15. Qin, W., et al., Near-infrared photocatalysis based on YF₃: Yb₃+,Tm₃+/TiO2 core/shell nanoparticles. Chemical Communications, 2010. 46(13): p. 2304-2306.
16. Yanes, A. C., et al., Novel Sol-Gel Nano-Glass-Ceramics Comprising Ln3+-Doped YF3 Nanocrystals: Structure and High Efficient UV Up-Conversion. Advanced Functional Materials, 2011. 21(16): p. 3136-3142.
17. Mahalingam, V., et al., Colloidal Tm3+/Yb3+—Doped LiYF4 Nanocrystals: Multiple Luminescence Spanning the UV to NIR Regions via Low—Energy Excitation. Advanced Materials, 2009. 21: p. 4025-4028.
18. Valérie Massardier, M. L., Photodegradation of a polypropylene filled with lanthanide complexes. Polímeros, 2015. 25(6): p. 515-522.
19. Chien, H.-W., et al., Interaction of LiYF4:Yb3+/Er3+/Ho3+/Tm3+@LiYF4:Yb3+upconversion nanoparticles, molecularly imprinted polymers, and templates. RSC Advances, 2020. 10(59): p. 35600-35610.
20. Gonell, F., et al., Aggregation-induced heterogeneities in the emission of upconverting nanoparticles at the submicron scale unfolded by hyperspectral microscopy. Nanoscale Advances, 2019. 1(7): p. 2537-2545.
21. Shah, S., et al., Hybrid upconversion nanomaterials for optogenetic neuronal control. Nanoscale, 2015. 7(40): p. 16571-16577.
22. Generalova, A. N., et al., Submicron polyacrolein particles in situ embedded with upconversion nanoparticles for bioassay. Nanoscale, 2015. 7(5): p. 1709-1717.
23. Hutten, I. M., CHAPTER 5—Processes for Nonwoven Filter Media, in Handbook of Nonwoven Filter Media, I. M. Hutten, Editor. 2007, Butterworth-Heinemann: Oxford. p. 195-244.
24. Sun, Y., et al., The biosafety of lanthanide upconversion nanomaterials. Chemical Society Reviews, 2015. 44(6): p. 1509-1525.
25. Mills, D., et al., Ultraviolet germicidal irradiation of influenza-contaminated N95 filtering facepiece respirators. Am J Infect Control, 2018. 46(7): p. e49-e55.
26. Buonanno, M., et al., Far-UVC light (222 nm) efficiently and safely inactivates airborne human coronaviruses. Scientific Reports, 2020. 10(1): p. 10285.
27. Lee, C., et al., Giant nonlinear optical responses from photon avalanching nanoparticles. arXiv preprint arXiv:2007.10551, 2020.
28. Zhou, B., et al., Enhancing multiphoton upconversion through interfacial energy transfer in multilayered nanoparticles. Nature Communications, 2020. 11(1): p. 1174.
29. Hernandez-Cordero, J., et al., Transparency power calculation in Yb3+-doped fiber due to temperature variations, in 2nd Workshop on Specialty Optical Fibers and Their Applications (WSOF-2). 2010.
30. Liao, L., et al., Can N95 Respirators Be Reused after Disinfection? How Many Times? ACS Nano, 2020. 14(5): p. 6348-6356.
31. Mackenzie, L. E., et al., The theoretical molecular weight of NaYF4: RE upconversion nanoparticles. Sci Rep, 2018. 8(1): p. 1106.
32. Sanderson, K., Quantum dots go large. Nature, 2009. 459: p. 760-761.
33. Jean, J., et al., Synthesis cost dictates the commercial viability of lead sulfide and perovskite quantum dot photovoltaics. Energy & Environmental Science, 2018. 11(9): p. 2295-2305.
34. Durmusoglu, E. G., et al., Low-Cost, Air-Processed Quantum Dot Solar Cells via Diffusion-Controlled Synthesis. ACS Applied Materials & Interfaces, 2020. 12(32): p. 36301-36310.
35. Bergman, M. S., et al., Impact of multiple consecutive donnings on filtering facepiece respirator fit. American Journal of Infection Control, 2012. 40(4): p. 375-380.
36. Boyda\check{g}, F. Ş., et al., Optical characterization of weakly absorbing PP, PE, and PP/PE films. Optics and Spectroscopy, 2003. 95(2): p. 225-229.
37. Yamada, K., et al., Equilibrium Melting Temperature of Isotactic Polypropylene with High Tacticity. 2. Determination by Optical Microscopy. Macromolecules, 2003. 36(13): p. 4802-4812.
38. Moan, J. and M. J. Peak, Effects of UV radiation on cells. Journal of Photochemistry and Photobiology B: Biology, 1989. 4(1): p. 21-34.
39. Peng, J., et al., Medical Waste Management Practice during the 2019-2020 Novel Coronavirus Pandemic: Experience in a General Hospital. American Journal of Infection Control, 2020.
40. Prata, J. C., et al., COVID-19 pandemic repercussions on the use and management of plastics. Environmental Science & Technology, 2020. 54(13): p. 7760-7765.

What is claimed is:

1. A wearable device, comprising:
an inner layer, an outer layer and a filter layer, wherein at least one of the inner layer, the outer layer or the filter layer includes one or more upconverting nanoparticles.

2. The wearable device of claim 1, wherein at least one of the inner layer, the outer layer or the filter layer further includes an ultra-violet (UV)-absorbing lanthanide complex material.

3. The wearable device of claim 2, wherein the UV-absorbing lanthanide complex material is configured to at least one of absorb UV radiation or down-convert the UV radiation back to infrared (IR) light.

4. The wearable device of claim 1, wherein the one or more upconverting nanoparticles include a NaYF₄ hexagonal host lattice.

5. The wearable device of claim 1, wherein one or more the upconverting nanoparticles are doped with Lanthanide ions.

6. The wearable device of claim 2, wherein the Lanthanide ions include a sensitizer to absorb near infrared photons and an emitter to send out photons at higher energies.

7. The wearable device of claim 6, wherein the sensitizer is Yb³⁺.

8. The wearable device of claim 6, wherein the emitter is Er³⁺ or Tm³⁺.

9. The wearable device of claim 1, wherein at least one of the inner layer or the outer layer includes non-woven polypropylene.

10. The wearable device of claim 1, wherein the filter layer is a meltblown filtration layer.

11. The wearable device of claim 1, wherein the one or more upconverting nanoparticles are provided in the filter layer.

12. The wearable device of claim 1, wherein the UV-absorbing lanthanide complex material is provided in the filter layer.

13. A method for disinfecting a wearable device, comprising:
providing the wearable device comprising an inner layer, an outer layer and a filter layer, wherein at least one of the inner layer, the outer layer or the filter layer includes one or more upconverting nanoparticles; and
applying infrared radiation (IR) to the one or more upconverting nanoparticles to generate ultraviolet (UV) radiation so as to disinfect the wearable device.

14. The method of claim 13, wherein the IR radiation includes the near-IR radiation.

15. The method of claim 14, wherein at least one of the inner layer, the outer layer or the filter layer further includes an ultra-violet (UV)-absorbing lanthanide complex material.

16. The method of claim 15, further comprising absorbing the UV radiation by the UV-absorbing lanthanide complex material upon the impact of the UV radiation on the wearable device.

17. The method of claim 15, further comprising down converting the UV radiation by the UV-absorbing lanthanide complex material into a subsequent IR radiation upon the impact of the UV radiation on the wearable device.

18. The method of claim 15, wherein the UV-absorbing lanthanide complex material is provided in the filter layer.

19. The method of claim 13, wherein the one or more upconverting nanoparticles are provided in the filter layer.

20. The method of claim 13, wherein one or more the upconverting nanoparticles are doped with Lanthanide ions.
